(19) United States
(12) Patent Application Publication
(10) Pub. No.: US 2023/0099033 A1
(43) Pub. Date: Mar. 30, 2023

(54) METHODS AND APPARATUS FOR A MITRAL VALVE ANNULOPLASTY RING DEVICE FOR VALVE-IN-RING TRANSCATHETER MITRAL VALVE REPLACEMENT

(71) Applicant: Hao Kenith Fang, Scottsdale, AZ (US)

(72) Inventor: Hao Kenith Fang, Scottsdale, AZ (US)

(21) Appl. No.: 17/944,428

(22) Filed: Sep. 14, 2022

Related U.S. Application Data
(60) Provisional application No. 63/249,265, filed on Sep. 28, 2021.

(51) Int. Cl. A61F 2/24

(52) U.S. Cl. CPC A61F 2/2448; A61F 2/2418; A61F 2250/006; A61F 2220/0075

(57) ABSTRACT

A valve-in-ring annuloplasty device according to the present technology includes a body coupled to an annuloplasty ring. The body is configured to align an artificial valve coaxially with the annuloplasty ring to reduce complications cause by misalignment of the artificial valve. A chamber wall, extending through the annuloplasty ring from a base portion configured to be attached to a valve annulus, is used to align the artificial valve during deployment. The chamber wall may include a plurality of struts configured to increase a rigidity of the chamber wall in the coaxial direction to ensure proper deployment of the artificial valve. The base portion may provide an increased suture area that decreases a likelihood for paravalvular leaking around the annuloplasty ring.

### CROSS-REFERENCES TO RELATED APPLICATIONS

This application claims the benefit of U.S. Provisional Patent Application No. 63/249,265, filed Sep. 28, 2021, and incorporates the disclosure of the application by reference.

### BACKGROUND OF THE TECHNOLOGY

Failed surgical mitral valve repairs are never anticipated but frequently encountered. As a result, patients often have to undergo an additional open heart surgery to either attempt a repair or more often a valve replacement. In 2021 the FDA approved of a method to replace the mitral valve in the setting of a failed prior mitral valve repair where an annuloplasty ring was utilized. The procedure is known as a valve-in-ring (VIR) transcatheter mitral valve replacement.

Referring now to FIG. 1, a prior art annuloplasty ring 100 may comprise a generally ring-shaped device intended to be positioned at the location where an artificial valve (not shown) will be installed. The annuloplasty ring 100 may form a continuous periphery or may be discontinuous such that the ring-shaped device has two ends separated by a small distance or gap. The artificial valve is positioned along an inner surface 102 of the annuloplasty ring 100 and connected to the annuloplasty ring 100. In the event of a failure of the repaired valve utilizing the annuloplasty ring 100, a new valve may need to be installed after the old annuloplasty ring 100 is removed. A less invasive procedure than open heart surgery may be used to replace the valve making use of the original annuloplasty ring 100.

More specifically, a VIR transcatheter mitral valve replacement involves the installation of a transcatheter heart valve within the original annuloplasty ring 100 through a percutaneous trans femoral venous approach. A catheter holds the replacement valve and is used to position the new valve inside of the original annuloplasty ring 100. A balloon may be inflated to locate and fix the position of the valve within the annuloplasty ring 100. This procedure reduces the need to perform a sternotomy or right thoracotomy and also eliminates the need for cardiopulmonary bypass. The procedure also eliminates the need for cardiac arrest and opening of the atrium of the heart to access the mitral valve.

Although the procedure is less invasive, there are complications associated with properly locating the new valve inside of the annuloplasty ring 100. For example, a common problem is the malposition of the valve within the original annuloplasty ring 100 due to the new valve not being aligned completely coaxially with the original annuloplasty ring 100 resulting in an off-axis installation. Other factors that impact a successful procedure include circularization of a D-shaped annuloplasty ring 100; paravalvular leakage between the original annuloplasty ring 100 and the new valve; paravalvular leakage around the annuloplasty ring 100 from dehiscence of the annuloplasty ring 100 from the native annulus of the mitral valve; and difficulties visualizing the location of the original annuloplasty ring 100 under fluoroscopy.

### SUMMARY OF THE TECHNOLOGY

A valve-in-ring annuloplasty device according to the present technology includes a body coupled to an annuloplasty ring. The body is configured to align an artificial valve coaxially with the annuloplasty ring to reduce complications caused by misalignment of the artificial valve. A chamber wall, extending through the annuloplasty ring from a base portion configured to be attached to a valve annulus, is used to align the artificial valve during deployment. The chamber wall may include a plurality of struts configured to increase a rigidity of the chamber wall in the coaxial direction to ensure proper deployment of the artificial valve. The base portion may provide an increased suture area that decreases a likelihood for paravalvular leaking around the annuloplasty ring.

### BRIEF DESCRIPTION OF THE DRAWINGS

A more complete understanding of the present technology may be derived by referring to the detailed description and claims when considered in connection with the following illustrative figures. In the following figures, like reference numbers refer to similar elements and steps throughout the figures.

FIG. 1 representatively illustrates a top view of a prior art annuloplasty ring;

FIG. 2 representatively illustrates a top view of an annuloplasty ring device in accordance with an exemplary embodiment of the present technology;

FIG. 3 representatively illustrates a side perspective view of the annuloplasty ring device shown in FIG. 2 in accordance with an exemplary embodiment of the present technology;

FIG. 4 representatively illustrates a top view of a mitral valve positioned within the annuloplasty ring device of FIG. 2 in accordance with an exemplary embodiment of the present technology;

FIG. 5 representatively illustrates a side view of the mitral valve positioned within the annuloplasty ring device in accordance with an exemplary embodiment of the present technology;

FIG. 6 representatively illustrates a side perspective view of an alternative embodiment of an annuloplasty ring device in accordance with an exemplary embodiment of the present technology;

FIG. 7 representatively illustrates a side perspective, partial phantom view of the alternative embodiment of an annuloplasty ring device shown in FIG. 6 in accordance with an exemplary embodiment of the present technology; and

FIG. 8 representatively illustrates a side partial phantom view of the alternative embodiment of an annuloplasty ring device shown in FIG. 6 in accordance with an exemplary embodiment of the present technology.

Elements and steps in the figures are illustrated for simplicity and clarity and have not necessarily been rendered according to any particular sequence. For example, steps that may be performed concurrently or in a different order are illustrated in the figures to help to improve understanding of embodiments of the present technology.

### DETAILED DESCRIPTION OF EXEMPLARY EMBODIMENTS

The present technology may be described in terms of functional block components and various processing steps. Such functional blocks may be realized by any number of components configured to perform the specified functions and achieve the various results. For example, the present technology may employ various materials, coupling mechanisms, dimensions, and geometries, which may carry out a variety of operations suited for attachment to or installation within a human heart. In addition, the technology described is merely one exemplary application for the technology. Further, the present technology may employ any number of conventional techniques for transcatheter valve placement.

Methods and apparatus for a valve-in-ring (VIR) annuloplasty device according to various aspects of the present technology may operate in conjunction with any type of artificial valve configured to be used with an annuloplasty ring, or VIR installation. Various representative implementations of the present technology may be applied to any type of valve replacement device that is insertable or otherwise intended for use within a body during a transcatheter procedure.

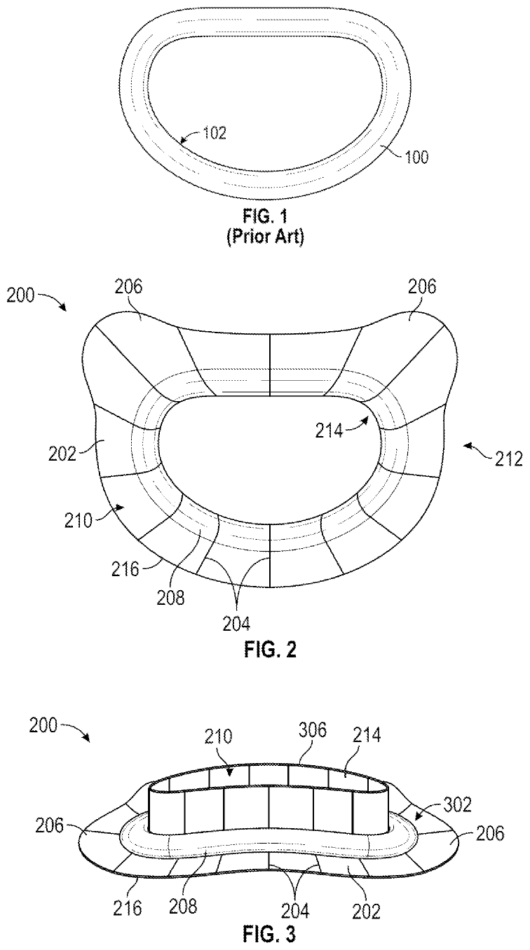

Referring now to FIGS. 2 and 3, in one embodiment a VIR annuloplasty device 200 may comprise a body 202 positioned around and coupled to a ring 208. The ring 208 may form a continuous or discontinuous (not shown) periphery around an open center that is configured to align with a flow path through the artificial valve. Similar to prior art annuloplasty rings, the ring 208 is configured to be positioned proximate a valve annulus and generally encircle the flow path through the heart valve. In an alternative embodiment, the annuloplasty device 200 may not include a ring 208 and may be configured to be coupled to a separate annuloplasty ring during the transcatheter procedure itself.

The body 202 may comprise a base portion and a chamber wall 214. An inner surface 210 of the body 202 may extend from a first edge 216 of the base portion to a second edge 306 of the chamber wall 214 Similarly, an outer surface 302 opposite the inner surface 210 may extend from the first edge 216 of the base portion to the second edge 306 of the chamber wall 214. The outer surface 302 may be positioned against and generally conform to the shape of the ring 208. Accordingly, the chamber wall 214, or inner core, itself may also match the shape of the ring 208. The chamber wall 214 is configured to protrude slightly into the atrium (or other chamber) above an annulus (not shown) where the annuloplasty device 200 is positioned.

A plurality of struts 204 may be located within the body 202 between the inner surface 210 and the outer surface 302 and extend around the periphery of the base portion and the chamber wall 214 between the first edge 216 of the body 202 and the second edge 306 of the body 202. The struts 204 may be configured to increase a rigidity of the chamber wall 214 of the body 202 along the axial (flow) direction. The increased rigidity provided by the struts 204 and the chamber wall 214 help align the replacement valve with the chamber wall 214 during placement of the replacement valve to decrease the likelihood that the replacement valve is positioned off-axis relative to the ring 208. For example, as a balloon positioned within the replacement valve is inflated the replacement valve expands within the open center of the annuloplasty device 200. As the replacement valve expands it engages the chamber wall 214 such that the outer walls of the replacement valve and the chamber wall 214 become coaligned. This allows the replacement valve to also be coaligned with the ring 208 such that the flow path through the replacement valve is more completely axial relative to the chambers on either side of the annulus and replacement valve.

The struts 204 may comprise a generally L-shaped configuration with a first side being aligned with a length of the chamber wall 214 and the second side being orthogonal to the chamber wall 214 and extending outwardly laterally away from the ring 208 in the base portion towards the first edge 216.

The body 202 may comprise any suitable size or shape and may be selected according to any applicable criteria, for example, the size of the ring 208 that the body 202 will be positioned over. For example, the first edge 216 of the body 202 may form an outermost lateral edge of the VIR annuloplasty device 200 that extends outwardly from the ring 208. The body 202 may extend outwardly from the ring 208 to help prevent leakage around or through the ring 208.

In one embodiment, the body 202 may comprise a pair of flaps 206 configured to be attached to the annulus of the atrial wall. The pair of flaps 206 may comprise a size and shape to overlap or at least partially conform to the commissures and trigones of the mitral valve. The surface area of the pair of flaps 206 may encourage the development of adhesions or scar tissue to form between the VIR annuloplasty device 200 and the native annulus and surrounding atrial tissue, thereby reducing a potential for paravalvular leakage to occur.

The body 202 may comprise any suitable material and may be configured to assist with the installation of the VIR annuloplasty device 200. For example, in one embodiment, the body 202 may comprise a suture permeable fabric material that can be sutured to the atrial wall. At least a portion of the body 202 along the chamber wall 214 may also comprise a radiopaque material that may allow for a visual depiction of the VIR annuloplasty device 200 so that a height that the chamber wall 214 extends into the left atrial chamber can be seen under fluoroscopy. For example, in one embodiment, the chamber wall 214 may comprise a height of about 1 centimeter and comprise a radiopaque material distributed around its entire periphery to help visualize how far the chamber wall 214 extends into the chamber.

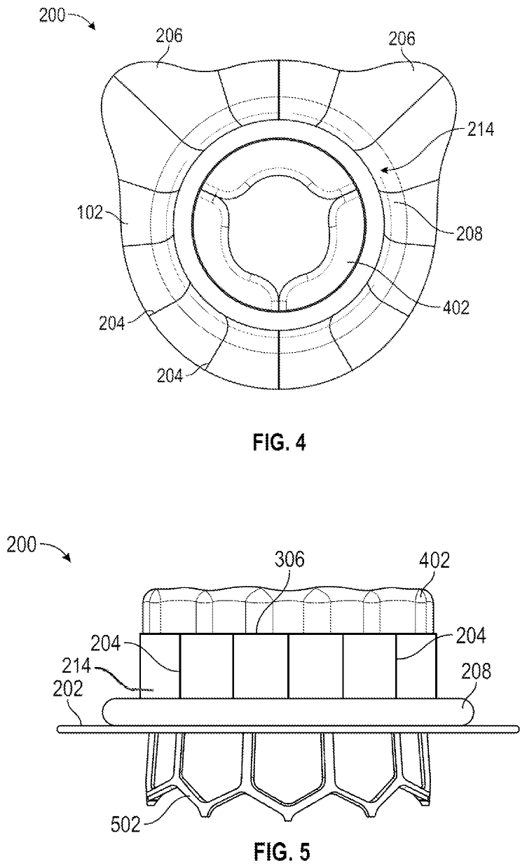

Referring now to FIGS. 4 and 5, the ring 208 and the body 202 may be configured to adjust in size to conform to shape of the replacement valve 402 after the replacement valve 402 is positioned within the chamber wall 214. For example, the ring 208 may comprise a D-shape when initially positioned or installed at the annulus but may be configured to conform to a generally circular shape of the replacement valve 402 and an associated frame 502. The plurality of struts 204 and the chamber wall 214 provide rigidity generally along the axial direction of the VIR annuloplasty device 200 while allowing the body 202 to be flexible in the radial direction to more completely conform to the shape of the replacement valve 402.

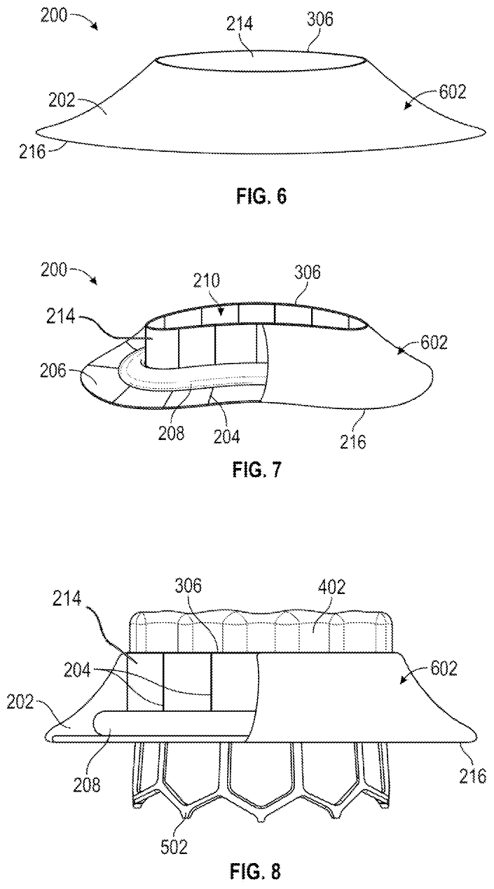

Referring now to FIGS. 6-8, The body 202 may further comprise a cover 602 for improving flow over the outer surface 302. For example, the cover 602 may extend from the second edge 306 to the first edge 216 to enclose the ring 208 within an interior of the body 202. The cover 602 may provide a smooth continuous surface between the second edge 306 and the first edge 216 to reduce a potential for clotting at or near the ring 208 that might be caused by disruptions to the flow impacting or otherwise engaging interface regions between the ring 208 and portions of the base and chamber wall 214 of the body 202.

The cover 602 may comprise any suitable material and may be formed of the same material as the rest of the body 202. The cover 602 may also be configured to be flexible or semi-rigid to help maintain a desired surface shape to improve the flow path over the top of the VIR annuloplasty device 200.

These and other embodiments for methods of creating an annuloplasty ring device may incorporate concepts, embodiments, and configurations as described above. The particular implementations shown and described are illustrative of the technology and its best mode and are not intended to otherwise limit the scope of the present technology in any way. Indeed, for the sake of brevity, conventional manufacturing, connection, preparation, and other functional aspects of the system may not be described in detail. Furthermore, the connecting lines shown in the various figures are intended to represent exemplary functional relationships and/or physical couplings between the various elements. Many alternative or additional functional relationships or physical connections may be present in a practical system.

The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the components and/or elements recited in any apparatus embodiment may be assembled or otherwise operationally configured in a variety of permutations to produce substantially the same result as the present technology and are accordingly not limited to the specific configuration recited in the specific examples.

As used herein, the terms “comprises,” “comprising,” or any variation thereof, are intended to reference a non-exclusive inclusion, such that a process, method, article, composition or apparatus that comprises a list of elements does not include only those elements recited but may also include other elements not expressly listed or inherent to such process, method, article, composition or apparatus. Other combinations and/or modifications of the above-described structures, arrangements, applications, proportions, elements, materials or components used in the practice of the present technology, in addition to those not specifically recited, may be varied or otherwise particularly adapted to specific environments, manufacturing specifications, design parameters or other operating requirements without departing from the general principles of the same. Any terms of degree such as “substantially,” “about,” and “approximate” as used herein mean a reasonable amount of deviation of the modified term such that the end result is not significantly changed. For example, these terms can be construed as including a deviation of at least ±5% of the modified term if this deviation would not negate the meaning of the word it modifies.

The present technology has been described above with reference to exemplary embodiments. However, changes and modifications may be made to the exemplary embodiments without departing from the scope of the present technology. These and other changes or modifications are intended to be included within the scope of the present technology, as expressed in the following claims.

1. An annuloplasty ring device for a valve replacement, comprising:
a ring forming a periphery around an open center configured to be positioned proximate a valve annulus;
a body coupled to the ring, comprising:
a base portion coupled to the ring and extending outwardly away from the open center and onto the valve annulus; and
a chamber wall extending away from the base portion along a flow direction through the open center of the ring.

2. An annuloplasty ring according to claim 1, wherein the body further comprises:
a first edge disposed along an outer perimeter of the base portion;
a second edge disposed along a perimeter of the chamber wall;
an inner surface extending from the first edge to the second edge;
an outer surface opposite the inner surface and extending from the first edge to the second edge; and
a plurality of struts disposed within the body between the inner surface and the outer surface and arranged around the perimeter of the chamber wall and extending from the first edge to the second edge.

3. An annuloplasty ring according to claim 2, wherein the body further comprises a cover extending from the second edge to the first edge over the outer surface to enclose the ring between the cover and the outer surface of the body.

4. An annuloplasty ring according to claim 1, wherein the chamber wall is oriented about ninety degrees to the base portion.

5. An annuloplasty ring according to claim 1, wherein the chamber wall forms a continuous surface between the periphery of the ring and the open center.

6. An annuloplasty ring according to claim 1, wherein the ring forms a continuous periphery around the open center.

7. An annuloplasty ring according to claim 1, wherein the ring forms a discontinuous periphery around the open center.

8. An annuloplasty ring according to claim 1, wherein the base portion comprises a pair of suture permeable flaps configured to align with a pair of trigones of the valve annulus.

9. An annuloplasty ring according to claim 1, wherein the body further comprises a radiopaque material disposed in at least a portion of the chamber wall.

10. An annuloplasty ring device for use with a replacement heart valve, comprising:
a ring forming a periphery around an open center configured to be positioned proximate a valve annulus, wherein:
the ring is formed of a semi-rigid material configured to conform to a circular shape of the replacement heart valve; and
the open center is aligned with a flow path through the replacement heart valve; and
a body coupled to the ring, comprising:
a base portion coupled to the ring and extending outwardly away from the open center and onto the valve annulus, wherein the base portion comprises a first edge disposed along an outer perimeter of the base portion;
a chamber wall extending away from the base portion along a flow direction through the open center of the ring, wherein the chamber wall:
comprises a second edge disposed along a perimeter of the axial wall portion; and
is configured to receive and align the replacement heart valve perpendicular to the base portion;
an inner surface extending from the first edge to the second edge;
an outer surface opposite the inner surface and extending from the first edge to the second edge; and
a plurality of struts disposed within the body between the inner surface and the outer surface and arranged around the perimeter of the chamber wall and extending from the first edge to the second edge.

11. An annuloplasty ring device according to claim 10, wherein the body further comprises a cover extending over the outer surface from the second edge to the first edge to enclose the ring between the outer surface of the body and the cover.

12. An annuloplasty ring device according to claim 10, wherein the chamber wall forms a continuous surface between the periphery of the ring and the open center.

13. An annuloplasty ring device according to claim 10, wherein the ring generally comprises a D-shape.

14. An annuloplasty ring device according to claim 13, wherein the ring forms a continuous periphery around the open center.

15. An annuloplasty ring device according to claim 13, wherein the ring forms a discontinuous periphery around the open center.

16. An annuloplasty ring device according to claim 10, wherein the base portion further comprises a pair of suture permeable flaps configured to align with a pair of trigones of the valve annulus.

17. An annuloplasty ring device according to claim 10, wherein the body further comprises a radiopaque material disposed in at least a portion of the chamber wall.

18. An annuloplasty alignment device for use with a replacement heart valve and an annuloplasty ring, comprising:
a body configured to be coupled to the annuloplasty ring along an outer surface, comprising:
a base portion extending outwardly away from an open center and onto an annulus of a heart valve, wherein the base portion comprises a first edge disposed along an outer perimeter of the base portion;
a chamber wall extending away from the base portion along a flow direction through the open center, wherein the chamber wall:
comprises a second edge disposed along a perimeter of the chamber wall; and
is configured to receive and align the replacement heart valve perpendicular to the base portion;
a plurality of struts disposed between an inner surface and the outer surface of the body and arranged around the perimeter of the chamber wall and extending from the first edge to the second edge; and
a cover extending from the second edge to the first edge to enclose the ring between the outer surface of the body and the cover.

19. An annuloplasty alignment device according to claim 18, wherein the base portion comprises a pair of suture permeable flaps configured to align with a pair of trigones of the valve annulus.

20. An annuloplasty alignment device according to claim 18, wherein the body further comprises a radiopaque material disposed in at least a portion of the chamber wall.
